(19) United States
(12) Patent Application Publication
(10) Pub. No.: US 2015/0061876 A1
(43) Pub. Date: Mar. 5, 2015

(54) Warning Device For Infusion Apparatus

(71) Applicant: Yi-Sheng Chang, Keelung City (TW)

(72) Inventor: Yi-Sheng Chang, Keelung City (TW)

(21) Appl. No.: 14/061,758

(22) Filed: Oct. 24, 2013

(30) Foreign Application Priority Data
Aug. 30, 2013 (TW) 102216301

(51) Int. Cl. A61M 5/168

(52) U.S. Cl. CPC A61M 5/16845
USPC 340/613

(57) ABSTRACT

A warning device for an infusion apparatus is installed at between a drip stand and a drip bottle, and the warning device includes an alarm module, a setting interface, a measurement module and a control module. The alarm module used to warn, the setting interface provides a total pharmaceutical dosage value, the measurement module measures the weight of the drip bottle with dosage to generate an original voltage value, and the control module connects with the alarm module, the setting interface and the measurement module respectively to read the total pharmaceutical dosage value and the original voltage value and to alarm by the alarm module. Therefore, the warning device solves the compatibility problem and able to apply in all kind of drip bottles such that the work loading and burden for the staff and the quality of medical care of the medical facility can be improved.

### FIELD OF THE INVENTION

The present invention relates to a warning device for an infusion apparatus, and particularly to a warning device for an infusion apparatus with an electronic scale therein for intravenous drip.

### BACKGROUND

In the modern medical, the intravenous injection is a common medical method. The intravenous injection is divided into a shortly injection and a continuity injection according to the injection time and injection volume, in which the continuity injection means an intravenous drip. The medical method of the intravenous drip is inserting the needle in the intravenous, and indwelling the needle at the intravenous, then the needle connects to one or more drip bottle with the dosage therein which can be replaced or added. The intravenous injection can let the drug of the drip bottle infuse to the intravenous of the patient slowly according to the gravity. In this way, the intravenous injection can provide a large number of the drug that the infusion requirement for the patient in one time.

According to aforementioned, during the intravenous injection used to infuse the drug into the patient, the drip bottle is to be replaced or added the dosage into the drip bottle when the dosage within the drip bottle is exhausted. However, the staff did not always monitor the residual dosage within the drip bottle and thus the warning device with a spring scale as a warning device to measure the weight, and the spring scale utilizes a spring stretching to be an alarm switch, when the drip bottle is to be exhausted and the weight of the drip bottle is to be light such that the warning device be turned on to alarm for reminding the user.

However, the elastic fatigue for the spring of the spring scale is inducted when the spring is used repeatedly. As this result, the spring scale will lose the original accuracy, and the staff cannot obtain the dosage of the drip bottle is run out at the first timing. Furthermore, the blood in the needle may also dry up such that the dosage cannot pass through the needle, so as to the needle is to be replaced to drip the drug into the patient. Thus, it will cause the pain for the patients and wasting the medical resources.

Therefore, a new mechanism or a new device is need to enable a warning device that use an electronic scale to sense the weight, and provide the staff can fine tuning the dosage through the accuracy of the electronic scale to increase the reliability and usage of the warning device.

### SOME EXEMPLARY EMBODIMENTS

According to above requirements, the mainly objective of the warning device for infusion apparatus of the present invention is to measurement dosage for injecting and to measure the weight of the drip bottle continuously. In addition, the residual dosage within the drip bottle is measured by measurement rule, and when the residual dosage within the drip bottle is too low, the warning device is to be alarmed. In this way, the warning device not only reduces the work loading and burden of the staff, but also improves the medical quality of the medical facilities.

According to the above purpose, a warning device for an infusion apparatus is installed at between a drip stand and a drip bottle, and the warning device includes an alarm module, a setting interface, a measurement module and a control module. The alarm module used to warn, the setting interface provides a total pharmaceutical dosage value, the measurement module measures the weight of the drip bottle with dosage to generate an original voltage value, and the control module electrically connects with the alarm module, the setting interface and the measurement module respectively to read the total pharmaceutical dosage value and the original voltage value and to alarm by the alarm module.

According to one embodiment of the present invention, the warning device further includes a power supply module. The power supply module electrically connects to the control module, and transmits a surplus power supply value to the control module.

According to one embodiment of the present invention, the alarm module further includes a visual alarm unit and an audible alarm unit.

According to one embodiment of the present invention, the setting interface includes an adjustment modify unit and a display unit. The adjustment modify unit electrically connects to the control module which provide a user for setting the total dosage value. The display unit electrically connects to the control module which displays the total dosage value. Furthermore, the adjustment modify unit has an increase key button and a decrease key button, and the display unit is a display panel.

According to one embodiment of the present invention, the measurement module is an electronic scale.

According to one embodiment of the present invention, the measurement module further connects to a hanging tool to hang the drip bottle.

### BRIEF DESCRIPTION OF THE DRAWINGS

The disclosure is illustrated by way of example, and not by way of limitation, in the figures of the accompanying drawings in which:

FIG. 1 is a structure chart of a warning device for infusion apparatus which is installed at between a drip stand and a drip bottle with the present invention;

FIG. 2 is a block diagram of a warning device for an infusion apparatus;

FIG. 3 is a flow chart of a measurement rule.

### DESCRIPTION OF THE PREFERRED EMBODIMENT

The embodiments of the apparatus and/or methods are disclosed. In the following description, for purposes of explanation, numerous specific details are set forth in order to provide a thorough understanding of the embodiments of the disclosure. It is apparent, however, to one skilled in the art that the present disclosure may be practiced without these specific details or with an equivalent arrangement.

The embodiments of the present disclosure are able to apply in all kinds of drip bottle.

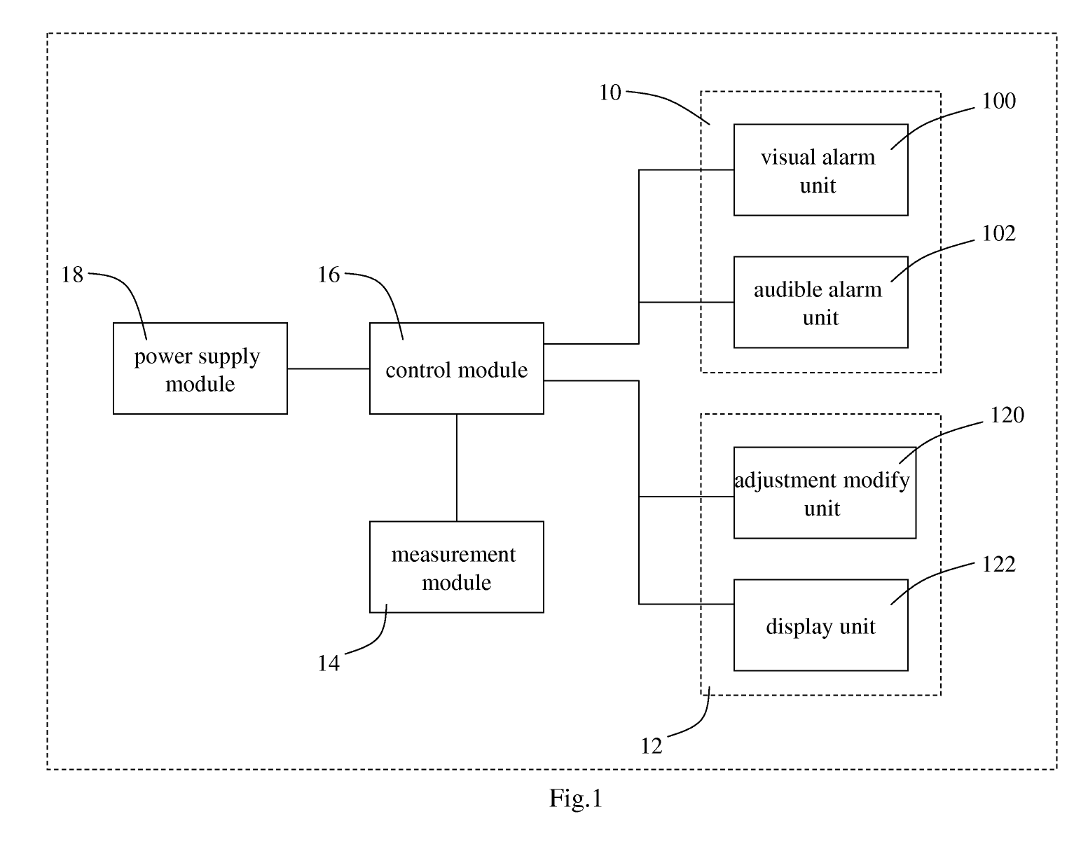

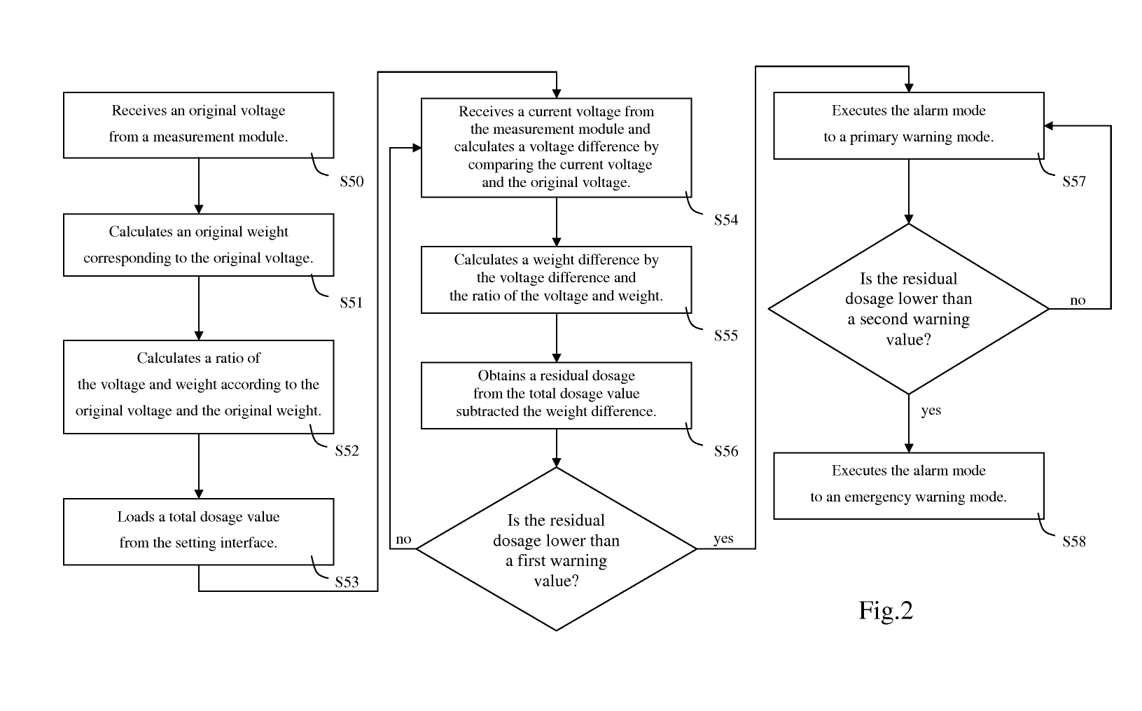

Please refer to FIG. 1 and FIG. 2. FIG. 1 is a structure view of a warning device for an infusion apparatus which is installed at between a drip stand and a drip bottle. FIG. 2 is a block diagram of a warning device for an infusion apparatus. In FIG. 1, a warning device for an infusion apparatus 1 is installed at between a drip stand 2 and a drip bottle 3, in which the warning device 1 monitors a dosage value within the drip bottle 3.

In FIG. 2, the warning device 1 includes an alarm module 10, a setting interface 12, a measurement module 14, a control module 16 and a power supply module 18, in which the control module 16 electrically connects to the alarm module 10, the setting interface 12, the measurement module 14 and the power supply module 18 respectively.

Then, the alarm module 10 includes a visual alarm unit 100 and an audible alarm unit 102, in which the visual alarm unit 100 is a light emission element such as a LED or a lamp to make the light warning, and the audible alarm unit 102 is a sound device such as a buzzer to make the audible warning. In addition, the alarm module 10 has a primary warning mode and an emergency warning mode. When the alarm module 10 is in the primary warning mode, the alarm module 10 will executes the visual alarm unit 100 and the audible alarm unit 102 every minute in a time interval, so that the alarm module 10 has one time warning every minute in the time interval. When the alarm module 10 is in the emergency warning mode, the alarm module 10 will starts the visual alarm unit 100 and the audible alarm unit 102 sustained in the time interval, so that the alarm module 10 has warns continuously in the time interval to attain a more strongly warning for the user.

The setting interface 12 is an operation platform for operating the warning device 1 which includes an adjustment modify unit 120 and a display unit 122. The user can set a total dosage value by using the adjustment modify unit 120 according to the dosage how much the user want to inject, and the total dosage value can be showed on the display unit 122 for the user. The display unit 122 may be a display panel, and the adjustment modify unit 120 may has an increase key button and a decrease key button for adjusting the total dosage value.

The measurement module may be an electronic scale, and further includes or connects to a hanging tool to hang the drip bottle 3 (as shown in the FIG. 1). Generally, the electronic scale includes a weight sensor therein and the electronic scale will apply a voltage to the weight sensor. When an analytic object applying a weight on the weight sensor, weight sensor is deformed which is caused by the gravity so as to the resistance of the weight sensor is to be changed and the voltage is also to be change. When there is a change in the weight of the analytic object, the voltage is also to be changed.

In other words, when the drip bottle 3 includes the drug therein on the hanging tool, the measurement module 14 will generate an origin voltage according to the weight of the drip bottle 3 which includes the drug therein. The measurement module 14 continuously monitors the voltage changed by changing the weight of the drip bottle 3 includes drug therein. With the dosage slowly drips into the patient's vein, the weight of the drip bottle 3 includes the dosage will be reduced. Thus, the measurement module 14 detects the voltage value which different from the original voltage value so as to a current voltage value is to be generated. It should be illustrated that in one infusion procedure, the initial voltage is defined as a constant value as the start of the infusion. Because there are many detection processes during the infusion process depended on the measuring frequency to generate a plurality of current voltage values.

The control module 16 electrically connects to the alarm module 10, the setting interface 12 and the measurement module 14 respectively. The control module 16 loads the total dosage value from the setting interface 12, and receives the original voltage value and the current voltage value from the measurement module 14 and according to the total dosage value, the original voltage value and the current voltage value that measured in accordance with measurement rules to execute the control module 10 for warning alert actions

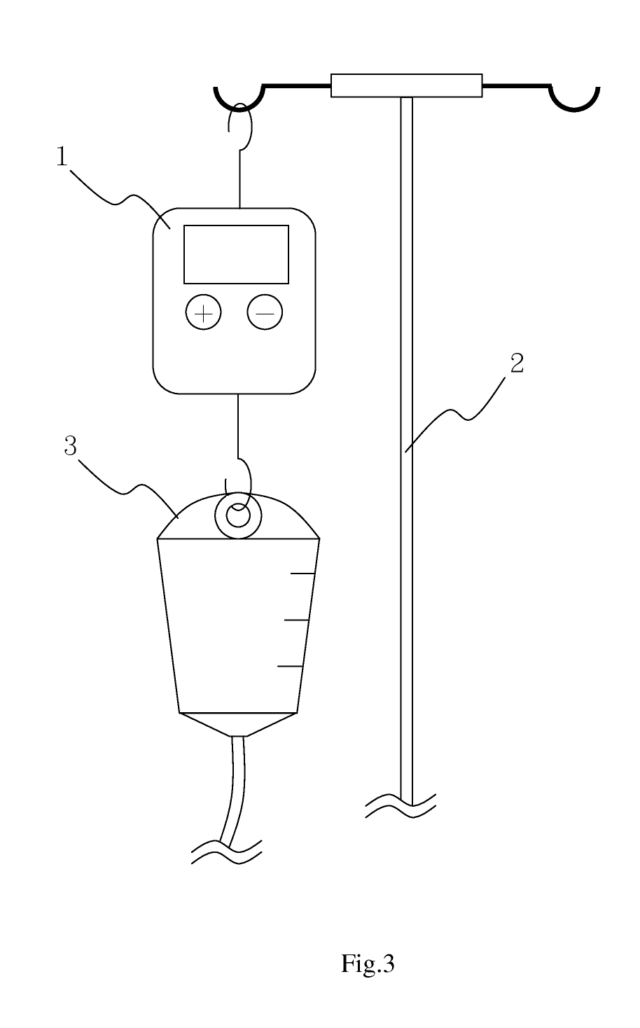

Refer to FIG. 3, which is a flow chart of a measurement rule. According to the warning device for an infusion apparatus 1 of the present invention, the measurement rule includes the step S50 denotes an original voltage that is received from a measurement module. The step S51 denotes an original weight that is calculated corresponding to the original voltage, and the original weight is the original weight of the drip bottle with the dosage therein. The step S52 denotes a ratio of the voltage and weight is calculated according to the original voltage and the original weight, and the ratio of the voltage and weight is a ratio value of the original voltage and the original weight. The step S53 denotes a total dosage value that is loaded from the setting interface. The step S54 denotes a current voltage is received from the measurement module and a voltage difference is calculated by comparing the current voltage and the original voltage. The step S55 denotes a weight difference is calculated by the voltage difference and the ratio of the voltage and weight, in which the ratio of the voltage difference and the weight difference are equal to the ratio value of the original voltage and the original weight, and the weight difference is the decrease of the drip bottle 3 when the starting of the infusion of the drip bottle 3, and the decrease of the drip bottle 3 is the amount of the infusion dosage. The step S56 denotes a residual dosage is obtained from the total dosage value subtracted the weight difference and when the residual dosage is lower than a first warning value, the alarm module is executed to a primary warning mode in the step S57. When the residual dosage is lower than a second warning value, the alarm module is executed to an emergency warning mode in the step S58, and the first warning value is larger than the second warning value.

For example, if the weight of 1 milliliter (ml) of the drug is 1 gram (g), the total weight of 200 ml dosage and drip bottle 3 with the dosage therein is 500 g and before the infusion treatment beginning, the measurement module 14 can detects an original voltage of the drip bottle 3 with the dosage therein which is denoted as the original voltage. The original voltage is transmitted to the control module 16 to calculate the original weight of the drip bottle 3 with the dosage therein is 500 g, which is the sum of the weight of the dosage and the drip bottle 3. Then, the ratio of the voltage and weight of the drip bottle 3 with the dosage therein is to be calculated in step S52, if the dosage of the infusion is 200 ml, the user can set the total dosage value is 200 ml through the setting interface 12 and the control module 16 can load the total dosage value is 200 ml from the setting interface 12.

Then, in a measurement frequency, when the infusion dosage is dipped out off the drip bottle 3 is 10 ml, the measurement module 14 will measure the voltage according to the reduction of 10 ml of the drip bottle 3 containing the dosage therein, and the voltage is the current voltage. At this time, the control module 16 compares the current voltage value with the original voltage in accordance with the step S54 to calculate the voltage difference and the weight difference is 10 g in accordance with the step S55. That is to say, the infusion of dosage is 10 ml. Thus, the residual dosage is 190 ml in the drip bottle 3 according to the step S56, and the residual dosage (190 ml) is larger than the first warning value such that the control module 16 did not execute the alarm module 10 to alarm.

How ever, with more and more of the dosage is dripped out off the drip bottle 3, the residual dosage within the drip bottle 3 is also fewer and fewer. When the residual dosage is lower than the first warning value, the control module 16 will executes the alarm module 10 to be the primary warning mode according to the step S57 to alarm the dosage is to be exhausted and to be replaced or supplement for the user. If the dosage is still dripped out off the drip bottle 3 and the user does not replace a new drip bottle 3 or supplement more dosage into the drip bottle 3, to let the residual dosage is lower than the second warning value such as 5 ml, the control module 16 transforms the alarm module 10 to an emergency warning mode from the primary warning mode to remind the dosage within the drip bottle 3 is to be run out for the user, whereby, by two-stages warning mechanism not only reduces the work loading and burden for the staff and improves the medical service quality of medical facilities can be improved by adding the alarm frequency.

Furthermore, the user may add the additional drug into the drip bottle 3 during the infusion process. At this time, the user can adjust the dosage within the drip bottle 3 by the adjustment modify unit 120 of the setting interface 12 after additional drug is added into the drip bottle 3. The added dosage is added to the total dosage value within the drip bottle 3. For example, if the additional drug is 50 ml, the total dosage value within the drip bottle 3 is adjusted to 250 ml according to above measurement rule. That is to say, the original dosage is 200 ml that is to be changed as the 250 ml, in this way, the additional dosage of the drug will not affect the original timing for warning, to achieve higher compatibility and higher maneuverability.

According to the above embodiments, the warning device 1 containing infusion apparatus further includes a power supply module 18. The power supply module 18 is the power supply for the warning device 1, and electrically connects to the control module 16. The control module 16 compares the surplus power supply value and a power warning value, when the surplus power supply value is lower than the power warning value, the control module 16 will instruct the alarm module 10 to alarm a lower battery message.

Certainly, the surplus power supply value can also display on the display unit 122 of the setting interface 12. Thus, the surplus power supply value of the power supply module can show for the user clearly and convenience for charging. Otherwise, the above alarm message can also show on the display unit 122 of the setting interface 12, and the residual dosage is calculated according to the step S56 within the drip bottle 3 can also be displayed on the display unit 122 of the setting interface 12 to show the residual dosage more clearly for the user.

Accordingly, the warning device can set the desired infusion dosage through the setting interface, the weight of the drip bottle is measured continuously by the measurement module, the residual dosage within the drip bottle is calculated according to the measurement rule of the control module and the control module executes the alarming when the residual dosage within the drip bottle is lower than the emergency warning value such that the work loading and burden for the staff and the quality of medical care of the medical facility can be improved.

While the invention has been described in connection with a number of embodiments and implementations, the invention is not so limited but covers various obvious modifications and equivalent arrangements, which fall within the purview of the appended claims. Although features of the invention are expressed in certain combination among the claims, it is contemplated that these features can be arranged in any combination and order.

What is claim is:

1. A warning device for an infusion apparatus is installed at between a drip stand and a drip bottle, the warning device comprising:
an alarm module;
a setting interface for providing a total dosage value;
a measurement module for measuring the weight of the drip bottle to generate an original voltage value; and
a control module for electrically connect to the alarm module, the set interface, and the measurement module to receive the total dosage value and the original voltage value and to alarm through the alarm module.

2. The warning device as claimed in claim 1, the warning device further comprising a power supply module, the power supply module electrically connects to the control module, and outputs a power to the control module for controlling the alarm module.

3. The warning device as claimed in claim 1, wherein the alarm module comprises a visual alarm unit and an audible alarm unit.

4. The warning device as claimed in claim 1, wherein the setting interface comprises:
an adjustment modify unit electrically connects to the control module for setting the total dosage value; and
a display unit electrically connects to the control module to display the total dosage value.

5. The warning device as claimed in claim 4, wherein the adjustment unit includes an increase key button and a decrease key button thereon.

6. The warning device as claimed in claim 4, wherein the display unit is a display panel.

7. The warning device as claimed in claim 1, wherein the measurement module is an electronic scale.

8. The warning device as claimed in claim 1, wherein the measurement module further connecting a hanging tool to hang the drip bottle.
